(19) United States
(12) Patent Application Publication
(10) Pub. No.: US 2023/0136651 A1
(43) Pub. Date: May 4, 2023

(54) METAL-PHOSPHORIZED CATALYST FOR PRODUCING 2,5-FURANDICARBOXYLIC ACID AND METHOD FOR PRODUCING 2,5-FURANDICARBOXYLIC ACID USING THE SAME

(71) Applicant: KOREA INSTITUTE OF SCIENCE AND TECHNOLOGY, Seoul (KR)

(72) Inventors: Dong Ki LEE, Seoul (KR); Jai Hyun KOH, Seoul (KR); Dahye WON, Seoul (KR); Ung LEE, Seoul (KR); Hyung-Suk OH, Seoul (KR); Byoung Koun MIN, Seoul (KR); JongIn WOO, Seoul (KR); Byeong Cheul MOON, Seoul (KR)

(21) Appl. No.: 17/840,633

(22) Filed: Jun. 15, 2022

(30) Foreign Application Priority Data
Nov. 2, 2021 (KR) 10-2021-0148900

(51) Int. Cl. B01J 27/185; C07D 307/68; B01J 37/02; B01J 37/04

(52) U.S. Cl. CPC B01J 27/1853; C07D 307/68; B01J 37/02; B01J 37/04

(57) ABSTRACT

According to one embodiment of the present invention, there is provided a catalyst compound, which comprises a compound of Chemical Formula 1 below and catalyzes the process of oxidizing 5-hydroxymethylfurfural (HMF) to 2,5-furandicarboxylic acid (FDCA):

NiCoₓP_{y} [Chemical Formula 1]

(wherein x and y are the molar ratio for Ni contained in the catalyst compound, 0<x<1, 0<y<1).

### TECHNICAL FIELD

The present invention relates to a metal-phosphorized catalyst for producing 2,5-furandicarboxylic acid and a method for producing 2,5-furandicarboxylic acid using the same.

### BACKGROUND ART

As consumption of petroleum-derived plastics continues to increase, greenhouse gas emissions for plastic production are projected to reach up to 15% of the global carbon budget by 2050. As a result, restrictions on the use of CO₂-free plastics composed of biomass-derived chemicals have continued to expand worldwide. Polyethylene furanoate (PEF) is a bio-based plastic that is gaining much attention due to its commercially attractive properties, such as high mechanical strength, excellent heat resistance, and excellent O₂ and CO₂ gas barrier properties, etc. The main component of PEF is 2,5-furandicarboxylic acid (FDCA). FDCA is considered as a platform chemical that can replace terephthalic acid in the production of various industrial polymers and chemicals.

FDCA can be produced from biomass, sugars, or platform chemicals such as 5-hydroxymethylfurfural (HMF) using biocatalytic transformation, aerobic oxidation, and electrochemical oxidation. Until now, the aerobic oxidation of sugars has been performed under high-temperature (approx. 140° C.) and high-pressure (approx. 40 bar) conditions using chemical oxidizing agents (O₂ and air) and noble metal catalysts (Pt, Pd, Au, and Ru). However, this conventional method has a problem in that a large amount of energy is consumed. Accordingly, there is an increasing need to develop an electrochemical production process that can be directly driven by a renewable energy source without energy loss.

### DISCLOSURE

### Technical Problem

An object of the present invention is to provide a metal-phosphorized catalyst for producing industrially useful 2,5-furandicarboxylic acid from biomass, and a production method therefor using the metal-phosphorized catalyst.

### Technical Solution

According to one embodiment of the present invention, there is provided a catalyst compound, which includes a compound of Chemical Formula 1 below and catalyzes the process of oxidizing 5-hydroxymethylfurfural (HMF) to 2,5-furandicarboxylic acid (FDCA):

NiCoₓP_{y} [Chemical Formula 1]

(wherein x and y are the molar ratio for Ni contained in the catalyst compound, 0<x<1, 0<y<1).

According to one embodiment of the present invention, there is provided the catalyst compound in which the amount of Ni³⁺ is higher than that of Ni²⁺ in the catalyst compound.

According to one embodiment of the present invention, there is provided a catalyst electrode, which includes a catalyst compound including a compound of Chemical Formula 1 below, and a substrate on which the catalyst compound is provided, and thereby catalyzes the process of oxidizing 5-hydroxymethylfurfural (HMF) to 2,5-furandicarboxylic acid (FDCA):

NiCoₓP_{y} [Chemical Formula 1]

(wherein x and y are the molar ratio for Ni contained in the catalyst compound, 0<x<1, 0<y<1).

According to one embodiment of the present invention, there is provided the catalyst electrode, in which the substrate is at least one selected from the group consisting of a metal foam, a metal foil, carbon paper, and carbon cloth.

According to one embodiment of the present invention, there is provided the catalyst electrode, in which nickel hydroxide is provided on the surface of the substrate.

According to one embodiment of the present invention, there is provided the catalyst electrode, in which the amount of Ni³⁺ is higher than that of Ni²⁺ in the catalyst compound.

According to one embodiment of the present invention, there is provided a process for producing FDCA, including: reacting 5-hydroxymethylfurfural (HMF) with a catalyst compound of Chemical Formula 1 below to oxide 5-hydroxymethylfurfural to 2,5-furandicarboxylic acid (FDCA):

NiCoₓP_{y} [Chemical Formula 1]

(wherein x and y are the molar ratio for Ni contained in the catalyst compound, 0<x<1, 0<y<1).

According to one embodiment of the present invention, there is provided the process for producing FDCA, in which 2,5-furandicarboxylic acid is produced by applying a potential of 1.40 V_{RHE} to 1.60 V_{RHE} to the 5-hydroxymethylfurfural (HMF) and the catalyst compound.

According to one embodiment of the present invention, there is provided the process for producing FDCA, in which the oxidation reaction of 5-hydroxymethylfurfural is performed in a basic environment without a base-induced polymerization reaction of the 5-hydroxymethylfurfural.

According to one embodiment of the present invention, there is provided an FDCA production reactor, including:

an inlet for introducing 5-hydroxymethylfurfural (HMF);

a catalyst electrode including a catalyst compound including a compound of Chemical Formula 1 below, and a substrate on which the catalyst compound is provided; and

an outlet for discharging 2,5-furandicarboxylic acid (FDCA) produced after the oxidation reaction of 5-hydroxymethylfurfural (HMF) performed in the catalyst electrode:

NiCoₓP_{y} [Chemical Formula 1]

(wherein x and y are the molar ratio for Ni contained in the catalyst compound, 0<x<1, 0<y<1).

According to one embodiment of the present invention, there is provided the FDCA production reactor, further including a power supply for applying a potential to the catalyst electrode.

According to one embodiment of the present invention, there is provided the FDCA production reactor, in which the substrate is at least one selected from the group consisting of a metal foam, a metal foil, carbon paper, and carbon cloth.

According to one embodiment of the present invention, there is provided the FDCA production reactor, in which nickel hydroxide is provided on the surface of the substrate.

According to one embodiment of the present invention, there is provided the FDCA production reactor, in which the amount of Ni³⁺ is higher than that of Ni²⁺ in the catalyst compound.

According to one embodiment of the present invention, there is provided a method for synthesizing a catalyst compound, including the steps of: preparing a NiCo bimetal compound by co-depositing Ni²⁺ and Co²⁺; and preparing a catalyst compound of Chemical Formula 1 below by reacting the NiCo bimetal compound with a phosphorus compound:

NiCoₓP_{y} [Chemical Formula 1]

(wherein x and y are the molar ratio for Ni contained in the catalyst compound, 0<x<1, 0<y<1).

According to one embodiment of the present invention, there is provided a method for synthesizing a catalyst compound, including the steps of:

mixing Ni²⁺, Co²⁺, a phosphorus compound, and a reducing agent; and

preparing a catalyst compound of Chemical Formula 1 below by heat treating the above mixture:

NiCoₓP_{y} [Chemical Formula 1]

(wherein x and y are the molar ratio for Ni contained in the catalyst compound, 0<x<1, 0<y<1).

### Advantageous Effects

The process of oxidizing 5-hydroxymethylfurfural (HMF) to 2,5-furandicarboxylic acid (FDCA) can be accelerated by the metal-phosphorized catalyst of the present invention.

Additionally, the metal-phosphorized catalyst of the present invention has excellent durability and thus can be repeatedly used for the production of 2,5-furandicarboxylic acid (FDCA).

### BRIEF DESCRIPTION OF DRAWINGS

FIG. 1 is a cross-sectional view showing the catalyst electrode according to one embodiment of the present invention.

FIG. 2 is a cross-sectional view schematically showing the FDCA production reactor according to one embodiment of the present invention.

FIG. 3a and FIG. 3b are flowcharts showing the method for synthesizing a catalyst compound according to one embodiment of the present invention.

FIG. 4 shows SEM analysis images of the catalyst electrodes according to Examples and Comparative Examples of the present invention.

FIG. 5 shows TEM analysis images of the catalyst electrodes according to Examples and Comparative Examples of the present invention.

FIG. 6 is a graph showing the results of XRD analysis of the catalyst electrodes according to Examples and Comparative Examples of the present invention.

FIG. 7 is an LSV graph analyzing the characteristics of the HMF oxidation reaction occurring in the catalyst electrodes according to Examples and Comparative Examples of the present invention.

FIG. 8 is a graph showing the results of ESCA analysis of the catalyst electrodes according to Examples and Comparative Examples of the present invention.

FIG. 9 is a graph showing the results of LSV analysis of the catalyst electrodes according to Examples and Comparative Examples of the present invention.

FIG. 10 is the results of XPS analysis of the catalyst electrodes according to Examples (NiCoP) and Comparative Examples (other than NiCoP).

FIG. 11 is the results of XPS analysis of the catalyst electrodes according to Examples (NiCoP) and Comparative Examples (other than NiCoP).

FIG. 12 is a graph showing the analysis results of oxidation reactions of HMF and intermediates using the catalyst electrodes of Examples and Comparative Examples.

FIG. 13 is a graph showing the analysis results of oxidation reactions of HMF and intermediates using the catalyst electrodes of Examples and Comparative Examples.

FIG. 14 is a graph showing the LSV and SSLSV analyses results of the catalyst electrodes according to Examples and Comparative Examples of the present invention.

### DETAILED DESCRIPTION OF PREFERRED EMBODIMENTS

The present invention will be described in more detail. The invention can have various modifications and take various forms, and thus specific embodiments are illustrated and described in detail below. It should be understood, however, that the invention is not intended to be limited to any particular disclosure form, but includes all modifications, equivalents, and alternatives falling within the spirit and scope of the invention.

In describing the drawings, like reference numerals are used for like elements. In the accompanying drawings, the dimensions of the structures may be enlarged compared to the actual dimensions for clarity of the invention. Although the terms “first”, “second”, and the like may be used herein to describe various elements, these elements should not be limited by these terms. These terms are only used to distinguish one element from another element. For example, a first element could be termed a second element, and similarly, a second element could be termed a first element, without departing from the scope of the present invention. Further, the singular forms are intended to include the plural forms as well, unless the context clearly indicates otherwise.

In the present application, it will be appreciated that terms “including” and “having” are intended to designate the existence of stated features, numbers, steps, operations, constituent elements, and components described in the specification or a combination thereof, and do not exclude the possibility of the existence or addition of one or more other features, numbers, steps, operations, constituent elements, and components, or a combination thereof in advance. Additionally, it will be understood that when an element such as a layer, film, region, or substrate is placed “on” or “above” another element, it indicates not only a case where the element is placed “directly on” or “just above” the other element but also a case where a further element is interposed between the element and the other element. Further, in the present specification, when an element such as a layer, film, region, or substrate is placed on another element, the formed direction is not limited to an upper direction, but a side and a lower direction may also be included. On the contrary, it will be understood that when an element such as a layer, film, region, or substrate is placed “beneath” or “below” another element, it indicates not only a case where the element is placed “directly beneath” or “just below” the other element but also a case where a further element is interposed between the element and the other element.

In the present specification, the terms “front side” and “back side” are used as relative concepts to facilitate the understanding of the inventive concept. Therefore, the “front side” and “back side” do not designate a specific direction, position, or constituent element and may be interchangeably used. For example, “front side” may be interpreted as “back side”, and “back side” may be interpreted as “front side”. Therefore, “front side” may be represented as “first side”, and “back side” may be represented as “second side”, while “back side” may be represented as “first side” and “front side” may be represented as “second side”. However, “front side” and “back side” are not used interchangeably with each other within a single embodiment.

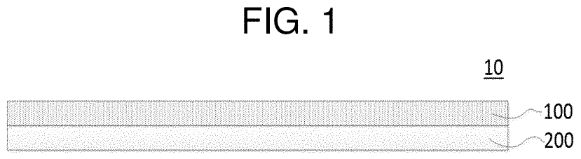

FIG. 1 is a cross-sectional view showing the catalyst electrode according to one embodiment of the present invention.

With reference to FIG. 1, there is provided a catalyst electrode 10, which includes a catalyst compound 100; and a substrate 200 on which the catalyst compound 100 is provided, and thereby catalyzes the process of oxidizing 5-hydroxymethylfurfural (HMF) to 2,5-furandicarboxylic acid (FDCA).

The catalyst compound 100 may have the composition of Chemical Formula 1 below:

NiCoₓP_{y} [Chemical Formula 1]

(wherein x and y are the molar ratio for Ni contained in the catalyst compound, 0<x<1, 0<y<1).

The catalyst compound 100 may be in a form in which phosphorus is uniformly doped into a bimetallic compound of Ni and Co. The catalyst compound 100 is composed of a combination of Ni, Co, and P as described above, so that FDCA can be prepared from HMF with high efficiency. In particular, the catalyst compound 100 may accelerate the oxidation reaction of HMF so that HMF can be oxidized into FDCA before the base-induced polymerization of HMF takes place even in a basic environment. The process of electrochemically producing FDCA from HMF is gaining attention as an alternative process because it uses mild reaction conditions (ambient pressure and temperature) and water as the oxygen source and co-produces hydrogen in the opposite reaction. Ni-based electrocatalysts such as Ni, Ni(OH)₂, Ni₃S₂, and Ni₂P have been developed for FDCA production of more than 90% in alkaline electrolytes, thereby achieving yield and Faraday efficiency (FE). Nevertheless, dissolved HMF under strongly basic conditions is very unstable because it can undergo base-induced polymerization. Therefore, the electrochemical performance of the HMF oxidation reaction is highly dependent on the OH⁻ ion concentration of the electrolytes. However, the catalyst compound according to the present invention can accelerate the HMF oxidation reaction such that HMF can be oxidized into FDCA before the base-induced polymerization of HMF takes place even in a basic environment.

Specifically, the catalyst compound 100 may have a Ni³⁺/Ni²⁺ ratio of 1.0 to 1.5, and may richly provide Ni³⁺ and Ni²⁺ on the surface of the catalyst electrode to improve the rate of all steps of the reaction from HMF to FDCA. In particular, as the amount of Ni³⁺ increases, the HMF oxidation reactivity may be greatly improved.

The above-described Ni³⁺/Ni²⁺ ratio of the catalyst compound 100 may be achieved by doping phosphorus into a Ni,Co bimetallic compound. By doping phosphorus into the Ni-based bimetallic compound, it can not only richly provide active ionic states (Ni²⁺, Ni³⁺) and finely modulate the electronic structure so as to minimize the activation energy of the intermediate reaction steps, but also, physical properties affecting the catalyst performance such as conductivity, defect density, and lattice distortion can be improved. These results are attributed to the hybridization of the anion sp orbital and the larger ionic radius compared to metal ions. For example, the catalytic activity can be accelerated as the Ni²⁺→Ni³⁺→Ni⁴⁺ state transition is accelerated.

The above-described advantageous effects of the catalyst compound 100 can be derived from the combination of Ni, Co, and P. It was confirmed that the reaction-accelerating effect was relatively poor when the Ni-based metal was mixed with metals other than Co or doped with S instead of P, etc. For example, the reactivity of sulfurized and phosphorized NiCu and NiFe electrodes was worse than that of NiS and NiP, respectively.

The relative ratios of Ni, Co, and P contained in the catalyst compound 100 may be 0.1 to 0.5 for Co and 0.1 to 0.5 for P with respect to 1 for Ni in an atomic ratio. Accordingly, the catalyst compound 100 may be provided in a Ni-rich state. As Ni, Co, and P are provided in the catalyst compound 100 within the above-described range, hybridization with the sp orbital between Ni or Co and P may take place while richly providing Ni. Therefore, Ni²⁺ and Ni³⁺ ion concentrations can be sufficiently secured in the catalyst compound 100.

The catalyst compound 100 may be provided on the substrate 200.

The substrate 200 supports the catalyst compound 100 and may function as a support member to allow electricity to pass through the catalyst compound 100.

The substrate 200 may be at least one selected from the group consisting of a metal foam, a metal foil, carbon paper, and carbon cloth. When the substrate 200 is a metal foam, the surface area of the substrate 200 is large, and when the catalyst compound 100 is applied on the substrate 200, the catalyst compound 100 may be provided on a relatively large area. Accordingly, the contact area between the catalyst compound 100 and the reactant is increased, thereby improving the efficiency of FDCA production reaction.

The substrate 200 may include Ni. Accordingly, nickel hydroxide may be provided on the surface of the substrate 200. A thin layer of nickel hydroxide may be provided on the surface of the substrate 200. When an anode potential is applied to the electrode to oxidize HMF, the nickel hydroxide layer on the surface of the substrate 200 may be converted into NiOOH in the form of Ni³⁺ hydroxide. Since the Ni³⁺ state is an important active site for the conversion of HMF to FDCA, the electrochemical oxidation of HMF can be mainly performed at the NiOOH site.

There is no limitation on the method of providing the catalyst compound 100 on the substrate 200. Accordingly, the catalyst compound 100 may be uniformly applied on the substrate 200 using conventional methods such as spray coating, etc.

The form of the substrate 200 may vary. The substrate 200 may have various forms such as a square, a rectangle, a circle, an ellipse, a triangle, a tripod, a rhombus, etc. on a plane. Since the form of the catalyst compound 100 applied and the form of the catalyst electrode 10 may be determined according to the form of the substrate 200, the form of the substrate 200 may be determined in consideration of the size, shape, capacity, etc. of the reaction apparatus.

As described above, the catalyst electrode 10 including the catalyst compound 100 and the substrate 200 may be provided in the FDCA production reactor.

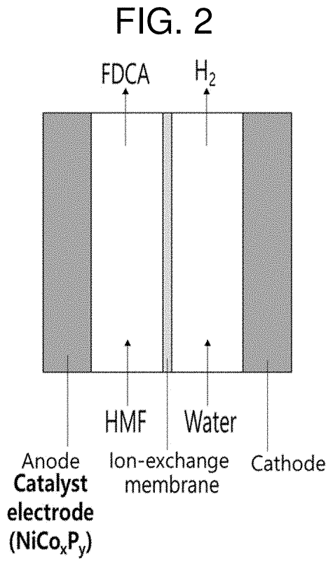

FIG. 2 is a cross-sectional view schematically showing the FDCA production reactor according to one embodiment of the present invention.

With reference to FIG. 2, the FDCA production reactor may include:

an inlet for introducing 5-hydroxymethylfurfural (HMF);

a catalyst electrode including a catalyst compound including a compound of Chemical Formula 1, and a substrate on which the catalyst compound is provided; and

an outlet for discharging 2,5-furandicarboxylic acid (FDCA) produced after the oxidation reaction of 5-hydroxymethylfurfural (HMF) performed in the catalyst electrode.

In describing each constitutional element of the FDCA production reactor, the catalyst electrode has previously been reviewed. In order to avoid duplication of content, the description thereof will be omitted below.

Inside the reactor, there may be provided a catalyst electrode and a cathode provided in a form that faces the catalyst electrode. The catalyst electrode may function as an anode in the reactor. An electrochemical reaction in which HMF is converted into FDCA may be performed by the catalyst electrode and the cathode.

The form and size of the anode (catalyst electrode) and the cathode may be determined in consideration of the capacity and form of the reactor. However, in some cases, the anode and the cathode may be provided in an extended form between the inlet through which the reactant is introduced and the outlet through which the product is discharged.

The inlet and outlet may be a region for introducing the reactants, i.e., HMF and water, into the reactor, and a region for discharging the reaction products, i.e., FDCA and hydrogen, to the outside of the reactor, respectively. The inlet and outlet may be integrated into one in some cases. For example, in the case of a batch reactor, the distinction between the inlet and the outlet may not be clear. A filter may be provided at each of the inlet and the outlet. The filter provided at the inlet and the outlet may perform a function of removing impurities mixed in the reactants and products.

The inlet and outlet may include an electromagnetic valve. Accordingly, the amount of reactants, the amount of products, the degree of reaction, etc. may be analyzed at the inlet and the outlet to adjust the reactant flow rate, etc.

A conductive separator may be further provided inside the reactor. The conductive separator separates the catalyst electrode from the cathode provided inside the reactor. Therefore, the electrochemical conversion reaction of HMF to FDCA may be performed on the catalyst electrode side of the reactor separated by the conductive separator, and the reaction by which water is converted into hydrogen may be performed on the cathode side of the reactor.

The FDCA production reactor may further be provided with a power supply for applying a potential to the catalyst electrode. The power supply may be connected to an external power source and the catalyst electrode, and may apply a potential necessary for the oxidation reaction of HMF.

Hereinafter, the conversion reaction of HMF to FDCA (FDCA production process) carried out in the above-described FDCA production reactor will be described in more detail.

The FDCA production process may be performed by reacting 5-hydroxymethylfurfural (HMF) with a catalyst compound of Chemical Formula 1 to oxidize HMF to 2,5-furandicarboxylic acid (FDCA). Intermediate species produced during the oxidative conversion of HMF to FDCA (5-hydroxymethyl-2-furancarboxylic acid (HMFCA), diformylfuran (DFF) and 5-formyl-2-furancarboxylic acid (FFCA)) may be formed. In the intermediate formation during FDCA production, DFF and HMFCA do not remain on the NiCoP electrode, and FDCA can be rapidly produced in proportion to the HMF conversion rate.

During the FDCA production process, a potential of 1.40 V_{RHE} to 1.60 V_{RHE} may be applied to the 5-hydroxymethylfurfural (HMF) and the catalyst compound to prepare 2,5-furandicarboxylic acid (FDCA). As described above, FDCA can be produced with high efficiency even under relatively mild reaction conditions, and this is because the catalyst compound used in the FDCA production process of the present invention has a very high reaction activity due to the combination of Ni, Co, and P.

Hereinafter, the method for preparing the above-described catalyst compound will be described.

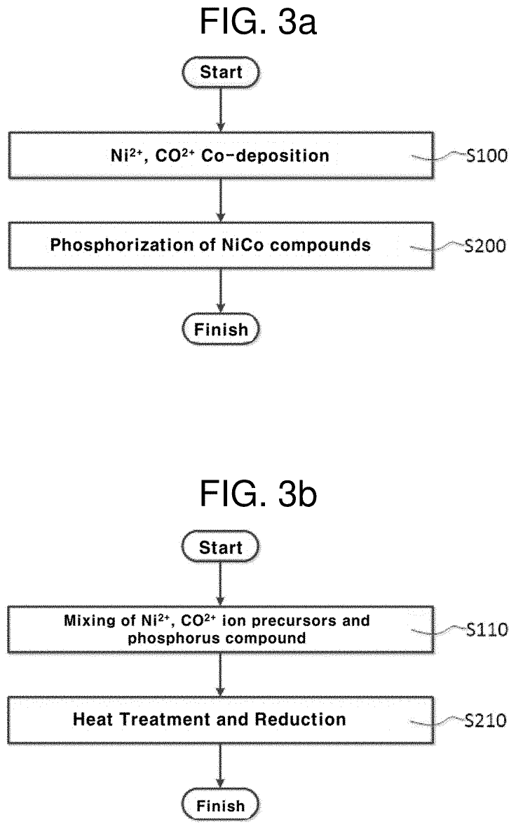

FIGS. 3a and 3b are flowcharts showing the method for synthesizing the catalyst compound according to one embodiment of the present invention.

With reference to FIG. 3a, the method for synthesizing a catalyst compound may include the steps of:

preparing a NiCo compound by co-depositing Ni²⁺ and Co²⁺ S100; and

preparing a catalyst compound of Chemical Formula 1 below by reacting the NiCo compound with a phosphorus compound S200.

First, in the step S100 of preparing a NiCo compound by co-depositing Ni²⁺ and Co²⁺, a Ni precursor and a Co precursor may be provided in a reactor including a working electrode, a counter electrode, and a reference electrode, and an electric potential may be applied to prepare the NiCo compound on the working electrode. The Ni precursor and the Co precursor may be any material without limitation as long as they are capable of providing Ni²⁺ and Co²⁺ ions, respectively. For example, NiSO₄.6H₂O and CoSO₄.7H₂O may be used as the Ni precursor and the Co precursor, respectively.

Subsequently, the step S200 of preparing a catalyst compound of Chemical Formula 1 by reacting the NiCo compound with a phosphorus compound may be performed by doping phosphorus into a bimetal compound or reacting a gaseous phosphorus compound with a bimetal compound.

With reference to FIG. 3b, in order to synthesize the catalyst compound, Ni²⁺, Co²⁺, and a phosphorus compound may be mixed S110, and the mixed solution may be heat-treated S210. In this case, NaH₂PO₂ may be used as the phosphorus compound. Additionally, a reducing agent may be further added in the solution for the reaction to proceed. As the reducing agent, ethylene glycol, etc. may be used. In the solution, the phosphorus compound may be dissolved to provide PO₂²⁻.

The mixed solution is then subjected to heat treatment S210, and the heat treatment may be performed in various ways. For example, the mixed solution may be heated using a microwave synthesizer. Alternatively, the heat treatment step S210 may be performed using a hydrothermal synthesizer. During the heat treatment process, PO₂²⁻ may bind Ni and Co ions to form a bond, and may be reduced by a reducing agent to synthesize NiCoP. The heat treatment temperature and time may vary depending on the amount of the precursor or phosphorus compound dissolved in the solution under the reaction conditions.

In the above, the catalyst compound, the catalyst electrode, the FDCA reactor, the FDCA production method, and the catalyst synthesis method according to one embodiment of the present invention have been described. Hereinafter, the advantageous effects of the catalyst compound according to the present invention will be described by way of Examples and Comparative Examples.

### Preparation Example 1. Synthesis of Catalyst Electrodes of Examples and Comparative Examples

NiCo, NiCu, and NiFe electrodes were prepared by electrodeposition of metal ions. The three-electrode cathodic electrodeposition using Ti foil as the working electrode, Pt mesh as the counter electrode, and Ag/AgCl (3 M NaCl) as the reference electrode was carried out by applying a constant potential of −1 V vs. Ag/AgCl and passing 1 C·cm⁻² through an aqueous solution containing 0.1 M NiSO₄.6H₂O and 0.3 M H₃BO₃. In order to synthesize the catalyst electrode of Comparative Examples, 0.01 M CoSO₄.7H₂O, 0.01 M CuSO₄, and 0.01 M FeSO₄.7H₂O were further added to the solution as precursors of Co, Cu, and Fe, respectively.

The thus-electrodeposited NiCo, NiCu, and NiFe were sulfurized by H₂S treatment. The Ni foil or the prepared Ni-based bimetallic electrodes were first calcined at 300° C. for 30 minutes to form metal oxides and then sulfurized in a tube furnace at 250° C. for 30 minutes in a H₂S gas atmosphere.

Next, NiP catalysts were synthesized by way of a hydrothermal method using a microwave synthesis reactor (Anton paar Monowave 400). 1 mmol of NiCl₂.6H₂O, 5 mmol of NaOH, and 10 mmol of NaH₂PO₂.H₂O were dissolved in 20 mL of ethylene glycol and 100 μL of deionized water (DI). Subsequently, 0.4 mmol of CoCl₂.6H₂O, 0.4 mmol of CuCl₂.2H₂O, and 0.2 mmol of FeCl₃.6H₂O were added to the mixture so as to synthesize NiCoP catalyst of the Example, and NiCuP and NiFeP catalysts of the Comparative Examples, respectively. The mixture was sonicated for 30 minutes to obtain a homogeneous solution, and then heated at 200° C. for 2 minutes with vigorous stirring at 600 rpm. After heat treatment, black precipitates were collected, washed by centrifugation with ethyl alcohol and deionized water, and dried in a vacuum oven for at least 3 hours.

Lastly, the catalysts of Examples and Comparative Examples prepared for electrochemical experiments were mounted on Ti foil or Ni foam. 8 mg of the catalysts was mixed with 0.375 mL of DI water, 0.125 mL of isopropyl alcohol, and 20 μL of Nafion solution, and the mixture was sonicated to prepare a uniform mixed solution. 300 μL of the prepared solution was spray coated on Ti foil (1 cm×2 cm), and 900 μL of the prepared solution was spray coated on Ni foam (1 cm×2 cm). The surfaces of the Ti foil and Ni foam were cleaned using ethyl alcohol and sulfuric acid before spray coating.

### Experimental Example 1. Analysis of Characteristics of Catalyst Electrodes of Examples and Comparative Examples

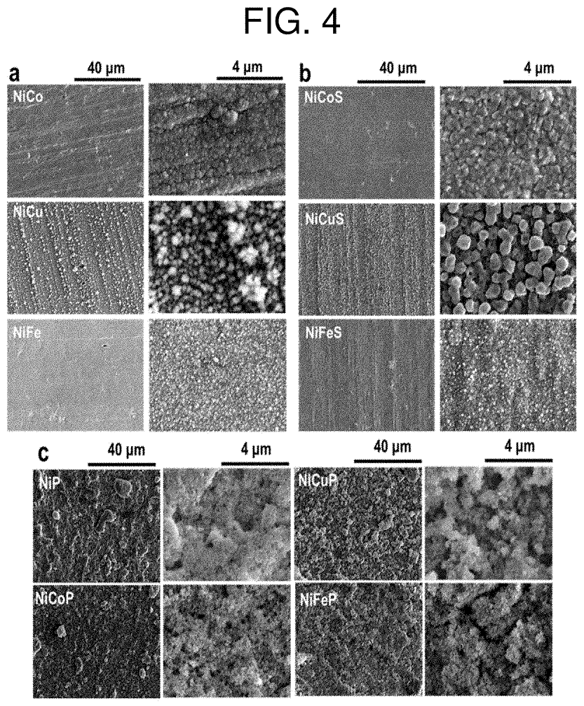

FIG. 4 shows SEM analysis images of the catalyst electrodes according to Examples and Comparative Examples of the present invention.

With reference to FIG. 4, the surface forms of the catalyst electrodes of the Examples (NiCoP) and Comparative Examples (NiCo, NiCu, NiFe, NiCoS, NiCuS, NiFeS, NiP, NiCuP, and NiFeP) can be confirmed.

The Ni-based bimetallic electrodes were prepared using co-deposition of Co²⁺, Cu²⁺, or Fe²⁺ ions with Ni²⁺. The applied potential and the amount of passing charge for the co-deposition of each electrode were determined by the performance evaluation of the electrochemical HMF oxidation. The form of the surface particles was similarly observed in NiCo, NiCu, and NiFe electrodes (FIG. 4, a). After sulfurization, the size of the surface particles was slightly enlarged due to aggregates during heat treatment at 300° C. (FIG. 4, b). The NiP, NiCoP, NiCuP, and NiFeP electrodes showed a rough surface form composed of extremely fine particles, as compared to the untreated and sulfurized electrodes (FIG. 4, c).

Next, the distribution of elements contained in the catalyst electrodes according to Examples (NiCoP) and Comparative Examples (NiCuP, NiFeP) was confirmed through TEM images.

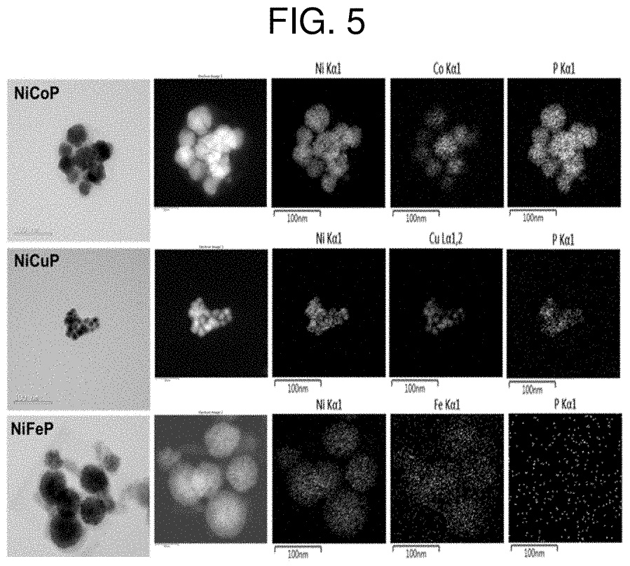

FIG. 5 shows TEM analysis images of the catalyst electrodes according to the Examples and Comparative Examples of the present invention.

The average particle size of the phosphorylated catalyst electrodes measured using transmission electron spectroscopy (TEM) was measured to be about 40 nm for NiCoP, 20 nm for NiCuP, and 70 nm for NiFeP (FIG. 5). The TEM energy dispersive spectroscopy (EDS) analysis showed that Ni, Co, Cu, Fe, and P elements were uniformly distributed throughout the particles.

Subsequently, the elemental composition of the catalyst electrodes of Examples (NiCoP) and Comparative Examples (NiCo, NiCu, NiFe, NiCoS, NiCuS, NiFeS, NiP, NiCuP, and NiFeP) was confirmed through XRD analysis.

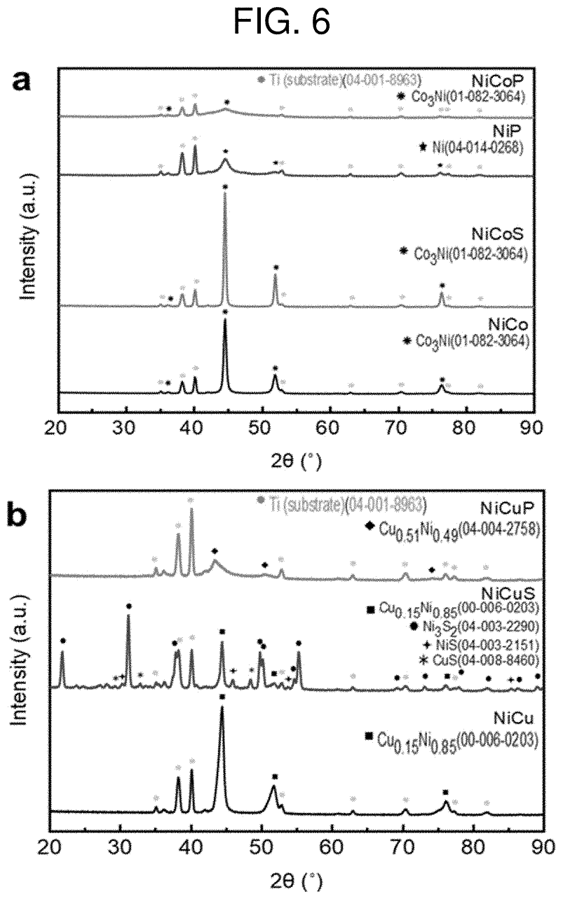

FIG. 6 is a graph showing the results of XRD analysis of the catalyst electrodes according to the Examples and Comparative Examples of the present invention.

The XRD analysis showed that the elemental composition was Co₃Ni (JCPDS 01-082-3064, FIG. 6, a) for total NiCo, Cu_{0.15}Ni_{0.85} (JCPDS 00-006-0203, FIG. 6, b) for NiCu, and Fe_{0.65}Ni_{0.35} (JCDPS 04-002-8939, FIG. 6, c) for NiFe. These alloy crystal structures were still maintained after sulfurization and phosphorization, but no crystalline metal chalcopyrite or phosphide structures were produced. However, the NiCuS spectrum exceptionally showed a strong peak corresponding to Ni₃S₂ (JCPDS 04-003-2290) and weak peaks corresponding to NiS (JCDPS 04-003-2151) and CuS (JCPDS 04-008-8460) with alloy NiCu crystals (FIG. 6, b).

The atomic ratios obtained from the SEM-EDS analysis indicated that NiCo and NiCu electrodes have a Ni-rich state, whereas NiFe has an Fe-rich state (Table 1). In contrast to the SEM-EDS analysis, the XPS analysis of the sulfurized electrodes showed a sulfur-rich state. Considering the different electron penetration depths between the SEM-EDS and XPS measurements, the sulfurized metals are expected to be distributed mostly on the particle surface. All atomic ratio analyses (SEM/TEM-EDS and XPS) of the phosphorylated electrodes showed a Ni-rich state as compared to the untreated and sulfurized electrodes. Considering that the relative proportion of phosphorus in the XPS analysis is lower than that in the SEM/TEM-EDS analysis, the phosphorized metals were mostly distributed in the particle core, which is a feature that is distinguished from the sulfurized electrodes.

TABLE 1

| Catalyst | | | |
|---|---|---|---|
| Electrode | SEM-EDS | XPS | TEM-EDS |
| NiCo | Ni:Co = 1:0.32 | — | — |
| NiCu | Ni:Cu = 1:0.30 | — | — |
| NiFe | Ni:Fe = 1:1.71 | — | — |
| NiS | Ni:S = 1:0.31 | 1:0.74 | — |
| NiCoS | Ni:Co:S = 1:0.31:0.05 | 1:0.16:0.79 | — |
| NiCuS | Ni:Cu:S = 1:0.29:0.34 | 1:5.52:2.61 | — |
| NiFeS | Ni:Fe:S = 1:1.01:0.04 | 1:2.03:3.93 | — |
| NiP | Ni:P = 1:0.17 | 1:0.20 | 1:0.40 |
| NiCoP | Ni:Co:P = 1:0.24:0.38 | 1:0.18:0.43 | 1:0.19:0.74 |
| NiCoP after | | 1:0.23:0.12 | 1:0.24:0.58 |
| Conversion of | | | |
| HMF to FDCA | | | |
| NiCuP | Ni:Cu:P = 1:0.50:0.29 | 1:0.11:0.11 | 1:0.69:0.61 |
| NiFeP | Ni:Fe:P = 1:0.60:0.65 | 1:0.00:0.33 | 1:0.30:0.04 |

### Experimental Example 2. Analysis of Characteristics of Electrochemical Conversion Reaction from HMF to FDCA

Next, the electrochemical conversion reaction from HMF to FDCA was analyzed in terms of how the reaction proceeded, in the catalyst electrodes according to Examples (NiCoP) and Comparative Examples (Ni foil, Co foil, Cu foil, Fe foil, Ni, NiCo, NiCu, NiFe, NiS, NiCoS, NiCuS, NiFeS, NiP, NiCuP, and NiFeP).

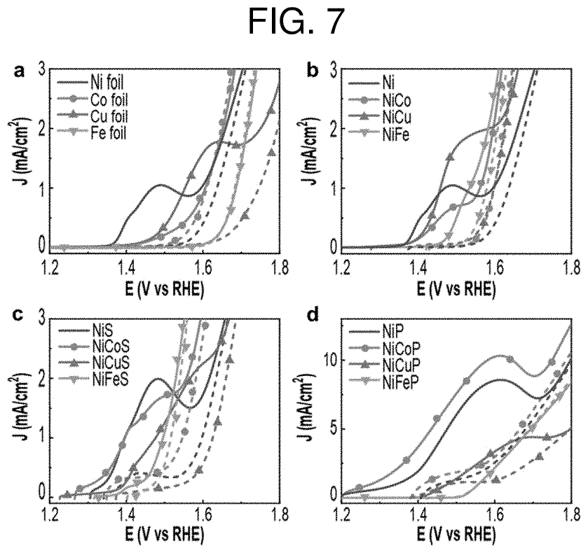

FIG. 7 is an LSV graph analyzing the characteristics of the HMF oxidation reaction occurring in the catalyst electrodes according to the Examples and Comparative Examples of the present invention.

Linear sweep voltammetry (LSV) curves of the Ni electrodes were obtained using Ni foil. A nickel hydroxide layer was first formed on the surface of the Ni foil by repeating the anodic LSV scan in KOH solution without HMF. The HMF oxidation current of the Ni foil electrode was generated at 1.35 V vs. RHE, and two oxidation waves were observed before the onset potential of the oxidation reaction of water at 1.53 V vs. RHE (FIG. 7, a).

The first current wave starting at the onset potential was mainly caused by the electrochemical regeneration of the Ni³⁺ state in the nickel hydroxide layer on the surface, which was reduced to the Ni²⁺ state to chemically oxidize the HMF. The second wave starting at about 1.40 V vs. RHE was the current generated by the electrochemical HMF oxidation reaction in the nickel hydroxide layer, which did not include the Ni²⁺/Ni³⁺ state transition.

The HMF oxidation properties of Cu, Co, and Fe foils were also investigated in the same manner. The bare Cu foil electrode also showed a definite oxidation wave for HMF conversion starting at 1.42 V vs. RHE, which was lower than the onset potential of water oxidation (1.60 V vs. RHE). While the HMF onset potential of the Cu foil was delayed as compared to that of the Ni foil, the diffusion-limited maximum current density (1.77 mA·cm⁻²) of the Cu foil before water oxidation took place was higher than that of the Ni foil (1.05 mA·cm⁻²). Co and Fe foils showed very small differences in current density between HMF and water oxidation reactions.

In order to investigate the binding effect of Ni with other metal ions, Ni-based bimetal electrodes (NiCo, NiCu, and NiFe) were prepared by electrodepositing Ni²⁺, Co²⁺, Cu²⁺, and Fe²⁺ ions on Ti foil (FIG. 7, b). When mixed with Ni, the HMF oxidation onset potential of the Cu electrode shifted closer to the oxidation onset potential of the Ni electrode (1.42 V to 1.35 V vs. RHE) while maintaining the diffusion-limited maximum current density of the bare Cu foil electrode (1.82 mA·cm⁻²). The HMF oxidation performance of the Co and Fe electrodes was improved by mixing with Ni, but was not as good as that of the Ni electrode. These results indicated that the Ni²⁺/Ni³⁺ state transition could be selectively accelerated by binding with specific metal ions.

In order to confirm the effect of the combination of anions on the transition of Ni ions, sulfurized and phosphorized catalyst electrodes were analyzed. The sulfurization of the electrodes was performed by H₂S gas treatment (FIG. 7, c). The maximum current density for HMF oxidation of Ni electrodes was increased by about 100% after sulfurization (NiS). However, in the case of NiCuS, the maximum current density for HMF oxidation after sulfurization was decreased as compared with the LSV curve of NiCu (FIG. 7, b).

Interestingly, among all bimetallic electrodes, NiCo showed a significant increase in the current density and onset potential at 1.27 V vs. RHE during HMF oxidation, and the Ni²⁺/Ni³⁺ transition characteristic of the LSV curve was clearly observed at 1.35 V vs. RHE. The HMF oxidation performance of NiCo was further improved by phosphorization. The NiCoP electrode showed a large increase of 10 mA·cm⁻² or more at the maximum current density, and the onset potential also significantly shifted to 1.20 V vs. RHE or less, showing superior characteristics than NiP (FIG. 7, d). In contrast, the HMF oxidation performance of NiCu was only slightly improved by phosphorization, and NiFeP showed no reactivity. These results indicated that the mixing with the anions manipulated the chemical state of Ni ions differently from the mixing with metal ions, suggesting that the selective combination of Ni with metal and anionic elements can greatly enhance the reactivity of electrochemical HMF oxidation.

Next, electrochemical active surface area (ESCA) analysis was performed on the catalyst electrodes according to Examples (NiCoP) and Comparative Examples (NiP, NiCuP, NiFeP) in order to confirm the effect of phosphorization of the bimetallic compounds on the surface area of the catalyst electrodes.

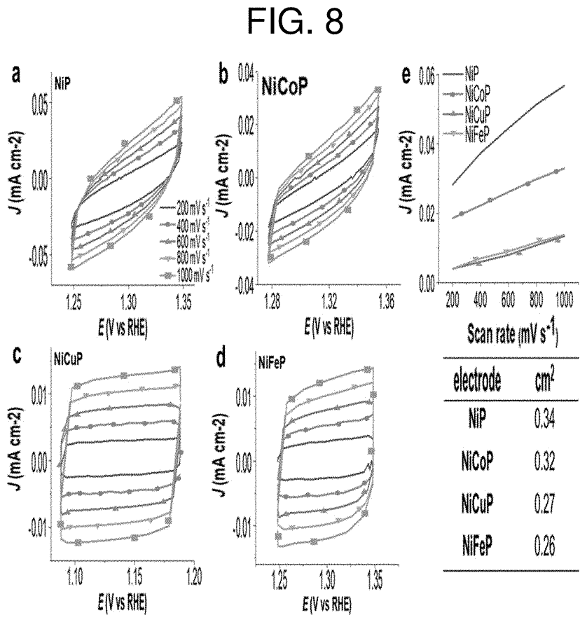

FIG. 8 is a graph showing the results of ESCA analysis of the catalyst electrodes according to the Examples and Comparative Examples of the present invention.

Since the electrochemical performance of the electrodes can be greatly improved by increasing the surface area without improving the intrinsic catalytic properties of the electrodes, the surface area of the phosphorized electrodes was compared using the electrochemically active surface area (ECSA) technique (FIG. 8). As a result of the analysis, NiP showed an ESCA value of 0.34 cm², which was the highest among all phosphorized electrodes. The ESCAs of the bimetallic electrodes (NiCoP, NiCuP, and NiFeP) were similar to each other (i.e., 0.26 cm² to 0.32 cm²), indicating that the superior performance of NiCoP was mainly attributed to the improved electrochemical catalytic properties for HMF oxidation.

The electrochemical conversion of HMF to FDCA was analyzed by applying an equivalent charge for complete conversion of HMF to FDCA at a constant potential within the divided cell. Since the onset potential and current density waves for water oxidation of HMF and all electrodes were not the same, the conversion of HMF to FDCA was performed at multiple potentials to obtain the optimal Faraday efficiency (FE) for each electrode. The results obtained at the potentials providing the highest yield and Faraday efficiency for FDCA production are listed in Table 2. Table 2 shows the results of the electrochemical conversion of 5 mM HMF to FDCA using the phosphorylated electrodes (foil and foam types) after passing a stoichiometric charge (40.52 C) at the applied potential of the optimal Faraday efficiency for FDCA production of each electrode.

TABLE 2

| | | Applied | HMF | HMFCA | DFF | FFCA | FDCA | FE for |
|---|---|---|---|---|---|---|---|---|
| | | potential | conversion | yield | yield | yield | yield | FDCA |
| Electrodes | | (∨_{RHE}) | (%) | (%) | (%) | (%) | (%) | (%) |
| Foil | NiCoP | 1.45 | 99.6 | 0.8 | — | 2.4 | 93.1 | 93.1 |
| | NiCuP | 1.60 | 90.4 | 0.4 | 2.5 | 13.9 | 58.6 | 58.6 |
| | NiFeP | 1.60 | 25.1 | 0.4 | 3.1 | 5.1 | 11.9 | 11.9 |
| | NiP | 1.50 | 99.3 | 0.3 | 0.6 | 13.8 | 76.8 | 76.8 |
| | NiCoS | 1.60 | 65.6 | 4.3 | 5.1 | 24.6 | 23.0 | 23.0 |
| | NiCuS | 1.55 | 52.3 | 1.4 | 3.2 | 7.2 | 39.4 | 39.4 |
| | NiFeS | 1.55 | 32.7 | 0.8 | 4.2 | 5.6 | 15.6 | 15.6 |
| | NiS | 1.60 | 85.4 | 4.2 | 2.8 | 28.4 | 40.5 | 40.5 |
| | NiCo | 1.55 | 33.1 | 2.9 | 1.2 | 4.1 | 12.5 | 12.5 |
| | NiCu | 1.55 | 74.4 | 0.9 | 1.3 | 3.2 | 63.4 | 67.4 |
| | NiFe | 1.60 | 26.3 | 1.7 | 2.6 | 6.6 | 11.4 | 11.4 |
| | Co | 1.50 | 78.2 | 7.2 | 3.3 | 36.3 | 22.0 | 22.0 |
| | Cu | 1.60 | 59.8 | 1.4 | 0.3 | 5.7 | 51.2 | 51.2 |
| | Fe | 1.70 | 13.4 | 1.3 | 1.9 | 2.6 | 2.2 | 2.2 |
| | Ni | 1.55 | 62.7 | 6.0 | 6.7 | 18.9 | 8.7 | 8.7 |
| Foam | NiCoP | 1.40 | 99.9 | — | — | — | 95.9 | 95.9 |
| | NiP | 1.45 | 99.9 | 0.5 | — | 17.6 | 80.7 | 80.7 |
| | Ni | 1.50 | 99.9 | 0.3 | — | 16.1 | 76.5 | 76.5 |

The Ni foil showed very poor results in terms of the yield and FE (8%) during the FDCA production. In addition, intermediate species (5-hydroxymethyl-2-furancarboxylic acid (HMFCA), diformylfuran (DEE), and 5-formyl-2-furancarboxylic acid (FECA)) produced during the oxidative conversion of HMF to FDCA remained in the anolyte even after passing the stoichiometric charge for complete conversion of HMF to FDCA (40.52 C).

The FDCA yield of Ni was gradually improved by sulfurization (NiS, 40%) and phosphorization (NiP, 76%). After complete conversion to FDCA, the amount of FECA (14%) similar to the Ni foil was retained in the anolyte of NiCoP, but only trace amounts of HMFCA (0.3%) and FECA (0.5%) were detected.

The Cu foil showed superior yield and FE for FDCA production (51%) among all foil electrodes of a single element. The FDCA production yield of Cu was further improved by more than 67% by the combination with Ni ions (NiCu). However, the FDCA production yield after sulfurization (NiCuS) and phosphorization (NiCuP) decreased sharply to 23% or 58%. Additionally, although the FDCA production yield of NiCo (12%) was lower than that of the Co foil (22%), the production yield was greatly improved by sulfurization (NiCoS, 23%) and phosphorization (NiCoP, 93%).

In contrast, all electrodes containing Fe had a production yield lower than the other electrodes in all cases. These results show that the FDCA production performance of the phosphorylated electrodes is significantly changed depending on the included metal ions compared to the bimetals or the sulfurized electrodes. Further, additional performance enhancement of NiP can be selectively achieved by mixing of Co ions.

The HMF oxidation reaction activity of the catalyst electrodes of Examples (NiCoP on Ni foam) and Comparative Examples (Ni on Ni foam, NiP on Ni foam) was analyzed by changing the substrate from flat foil to porous nickel foam.

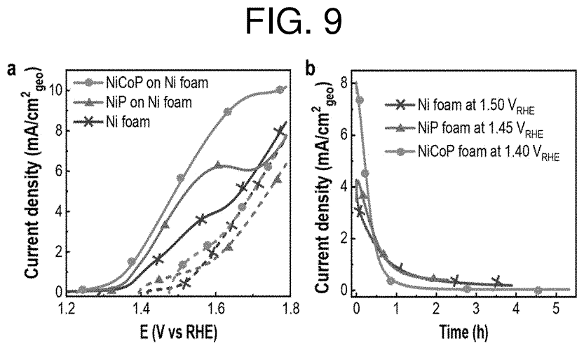

FIG. 9 is a graph showing the results of LSV analysis of the catalyst electrodes according to the Examples and Comparative Examples of the present invention.

The HMF conversion test was repeated with a foam-type electrode that can easily increase current density due to its high specific surface area while maintaining the intrinsic characteristics of the catalyst. Commercial Ni foam and NiP or NiCoP foam electrodes used for Ni were prepared by spray coating NiP or NiCoP nanoparticles on the commercial Ni foam. The LSV scans of the Ni, NiP, and NiCoP foam electrodes showed the same performance trends as those of the foil electrodes (FIG. 9a).

During the constant potential oxidation, the commercial Ni foam electrode showed a higher current density than Ni foils. Thus, the charge of 40.52 C was able to pass within 4 hours at the applied potential of 1.50 V vs. RHE (FIG. 9b). The optimal Faraday efficiency for FDCA production of Ni increased from 8% to 76% when the foil was replaced with the foam type. The charge efficient conversion from HMF to FDCA was also achieved using NiP and NiCoP foam electrodes at lower applied potentials than the foil type.

The NiCoP foam electrode between NiP and NiCoP showed a much higher starting current density (approx. 8 mA·cm⁻²) than the commercial Ni foam (approx. 4 mA·cm⁻², FIG. 9b), and showed almost complete conversion from HMF to FDCA at 1.40 V vs. RHE with no reaction intermediate residues.

The FDCA yield of 95% and Faraday efficiency at 1.40 V vs. RHE by the NiCoP form electrode was significantly higher than those of the catalysts according to the prior art studied previously at pH 13 (0.1 M KOH) (Table 3).

TABLE 3

| | Onset | Maximum | | | |
|---|---|---|---|---|---|
| | potential | current | | | |
| | for HMF | density | HMF | FDCA | FE for |
| Catalyst/ | oxidation | before OER | Conc. | yield | FDCA |
| Substrate | (V_{RHE}) | (mA · cm⁻²) | (mM) | (%) | (%) |
| NiCoP/Ni foam | 1.22 | 5.5 at 1.50 | 5 | 95.9 at | 95.9 |
| | | V_{RHE} | | 1.40 V_{RHE} | |
| NiBₓ—P_{0.07}/ | 1.40 | 1.1 at 1.46 | 10 | 90.6 at | 92.5 |
| Carbon | | V_{RHE} | | 1.45 V_{RHE} | |
| paper[1] | | | | | |
| 2D MOF | 1.45 | 5.0 at 1.55 | 10 | 99.0 at | 78.8 |
| NiCoBCD/Ni | | V_{RHE} | | 1.55 V_{RHE} | |
| foam[2] | | | | | |
| Nano Cu foam/ | 1.25 | 3.1 at 1.60 | 5 | 96.4 at | 95.3 |
| Cu foil[3] | | V_{RHE} | | 1.62 V_{RHE} | |
| NiOOH/FTO[4] | 1.25 | 1.1 at 1.47 | 5 | 96.0 at | 96.0 |
| | | V_{RHE} | | 1.47 V_{RHE} | |
| Pd₁Au₂/C/ | 0.48 | 7.0 at 0.87 | 20 | 83.0 at | — |
| Glassy carbon | | V_{RHE} | | 0.90 V_{RHE} | |
| cloth[5] | | | | | |

Additionally, even after the complete conversion from HMF to FDCA was repeated five times at a constant potential, the FDCA yield and FE of the NiCoP foam were maintained at 93% or more, indicating that NiCoP had excellent reaction stability. The HMF oxidation current of the NiCoP foam electrode decreased sharply from 10 mA·cm⁻² to less than 200 μA·cm⁻² within one hour (FIG. 9b), and most of the intermediate conversion to HMF and FDCA was completed in the first step due to the batch reaction conditions (the amount of reactants was limited). A longer reaction time was required in order to pass the stoichiometric charge to NiCoP than to Ni and NiP foam electrodes because very little reactive species remained in the anolyte after the first hour.

Although the problem of the base-induced HMF polymerization was not completely resolved even in a 0.1 M KOH solution, the initial concentration of HMF (5 mM) was almost maintained (<97%) during the first 2 hours, and 99% of the initial HMF concentration was remained even after the first 2 hours, when the HMF concentration was low (0.5 mM). As can be seen in FIG. 9b, when the foam-type NiCoP electrode was used, most of the HMF conversion was completed during the first hour, and thus, the problem of HMF decomposition in 0.1 M KOH solution was negligible.

### Experimental Example 3. XPS Analysis Results of NiCoP Catalyst Electrode

Next, the chemical state of Ni ions affected by the binding of metal ions with S/P anions was investigated through X-ray photoelectron spectroscopy (XPS).

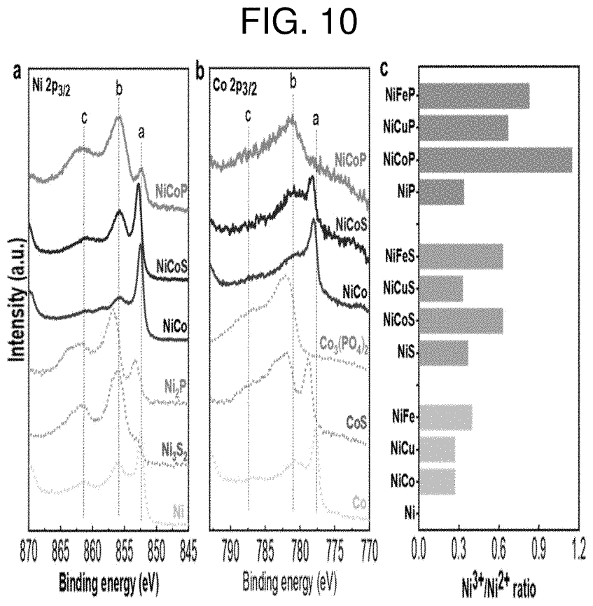

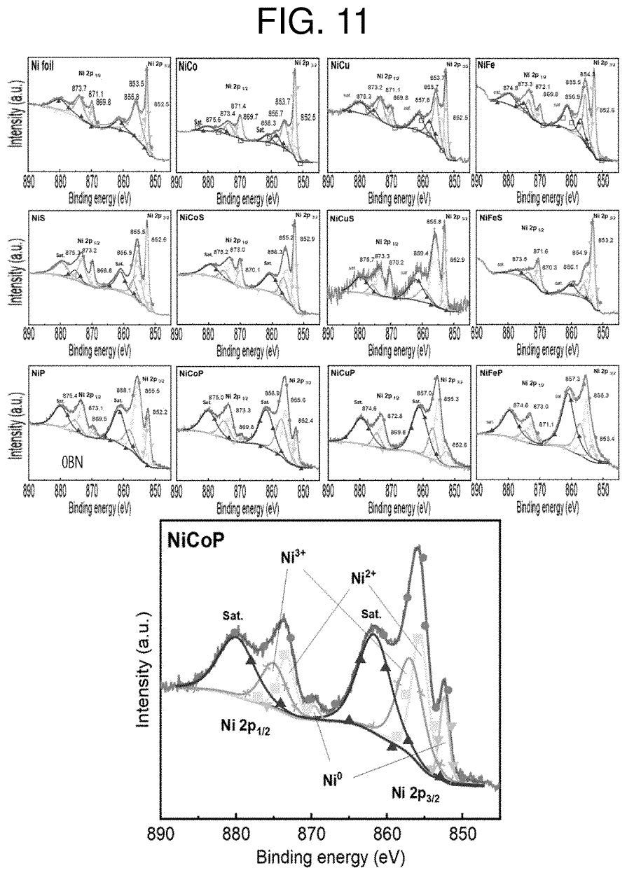

FIGS. 10 and 11 are XPS analysis results of the catalyst electrodes according to Examples (NiCoP) and Comparative Examples (other than NiCoP).

When the Ni 2p_{3/2} spectrum of the Ni foil was compared with the Ni₃S₂ and Ni₂P reference, the peak corresponding to the metallic Ni state (Ni⁰, the peak in FIG. 10, a) gradually shifted to higher bonding energy as Ni was bonded to S and P. In addition, the relative intensity ratio of the b peaks (Ni²⁺+Ni³⁺) and (Ni⁰) gradually increased from Ni to Ni₃S₂ and Ni₂P, changing the intensity and form of the c peak (Ni 2p_{3/2} satellite). These features were observed in the Ni 2p_{3/2} spectrum of the prepared NiCo, NiCoS, and NiCoP electrodes. Among the prepared electrodes, NiCoP showed significant differences in the Ni²⁺+Ni³⁺ (b) and Ni 2p_{3/2} satellite (c) peaks compared to those of NiCo and NiCoS, which proved that a large amount of Ni²⁺ and Ni³⁺ states were generated by phosphorylation treatment. Further, the Ni⁰ peak (a) of NiCoP exceptionally showed a tendency opposite to that of the Ni₂P reference. The Ni⁰ peak of NiCoP shifted to a lower binding energy close to that of Ni metal, whereas the peak of Ni₂P reference shifted to a higher binding energy than that of Ni₃S₂ and Ni. As can be seen from the energy dispersive spectroscopy (EDS) analysis results, it was estimated that the NiCoP and NiP electrodes have a Ni-rich state compared to the Ni₂P reference. The presence of the Ni-rich state had an effect to the Ni⁰ state of the phosphorylated Ni species, leading to a shift to a lower binding energy. The same characteristics as the Ni 2p spectrum were observed in the Co 2p spectrum of NiCoP. The intensity of the Co²⁺+Co³⁺ peak (b) of NiCo was gradually increased. However, the metallic Co peak (a) was reduced by sulfurization and phosphorization (FIG. 10b), indicating that phosphorization was the most effective way to simultaneously generate Ni²⁺ and Ni³⁺ states as well as Co²⁺ and Co³⁺ states.

The Ni 2p spectrum of NiCuP and NiFeP were similar to the Ni 2p spectrum of NiCoP. Since Ni³⁺ was the most reactive to HMF oxidation, the relative ratio of Ni³⁺ vs. Ni²⁺ was quantitatively compared by deconvoluting the XPS Ni 2p peaks of all electrodes (FIG. 11). In the bimetallic electrodes, the ratio of Ni³⁺/Ni²⁺ was gradually increased by sulfurization and phosphorylation (FIG. 10c). Among the bimetal electrodes, NiCoP had the highest Ni³⁺/Ni²⁺ ratio (1.15), which indicated that a Ni³⁺-rich state existed on the NiCoP surface. The XPS analysis showed that the anion incorporation substantially alters the chemical state of Ni ions. Moreover, the phosphorization of the bimetallic electrodes significantly increased the oxidation state of the metal ions as compared to sulfurization.

### Experimental Example 4. Electrochemical Oxidation Characteristics of HMF and Intermediates by Catalysts

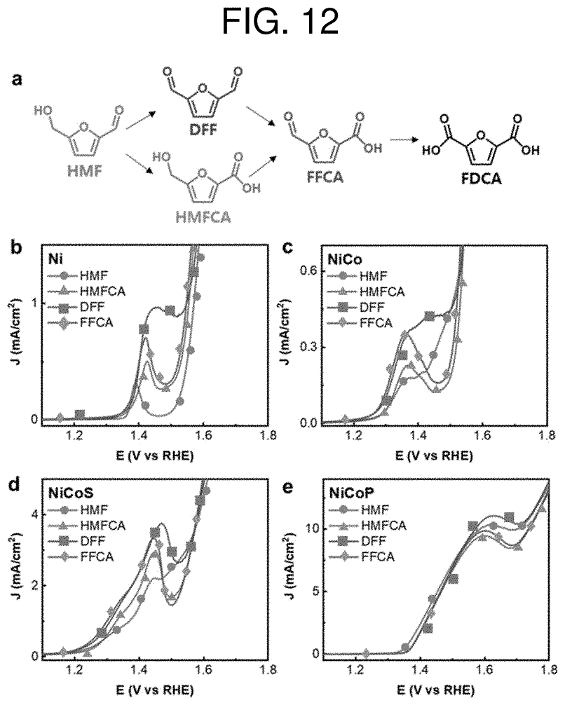

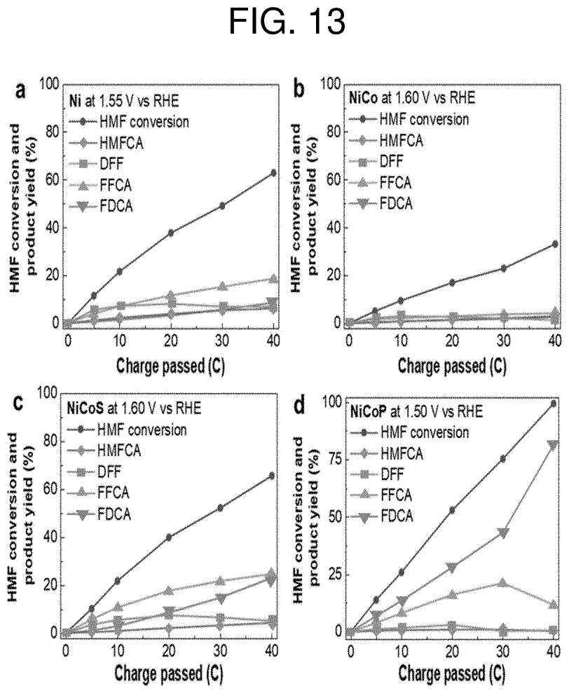

Next, the reactivity of NiCo-based electrodes in the HMF-derived molecular oxidation was investigated. FIGS. 12 and 13 are graphs of analysis results of oxidation reactions of HMF and intermediates using the catalyst electrodes of Examples and Comparative Examples.

FIG. 12 shows the LSV curves for the oxidation of 5 mM HMF and intermediates (DFF, HMFCA, and FFCA), which are the results obtained by performing oxidation using (b) Ni, (c) NiCo, (d) NiCoS, and (e) NiCoP foil electrodes in a 0.1 M KOH solution with a scan rate of 10 mV·s⁻¹. FIG. 12 is a result of confirming the conversion yield of HMF in a KOH solution at pH 13 using (b) Ni, (c) NiCo, (d) NiCoS, and (e) NiCoP foil electrodes.

The onset potential (1.34 V vs. RHE) of HMF oxidation in the Ni foil was higher than that of possible intermediates (DFF: 1.30 V vs. RHE, HMFCA: 1.31 V vs. RHE, and FFCA: 1.25 V vs. RHE), and the maximum current density for the HMF oxidation was the lowest among all oxidation reactions (FIG. 12, b). These results show that HMF oxidation, which was the initial step, was the most difficult reaction for Ni among all conversion steps. The characteristics of delayed onset and low current density for HMF oxidation were also observed in NiCo (FIG. 12, c) and NiCoS (FIG. 12, d) electrodes. Although the onset potential of HMF oxidation was shifted closer to that of HMFCA oxidation, the maximum current density of HMF oxidation was still lower than that of HMFCA, indicating that the initial HMF oxidation step was the reaction determining step for FDCA production in the Ni, NiCo, and NiCo electrodes. In contrast, the NiCoP electrode showed the fastest onset potential for HMF oxidation, and the maximum current density of HMF oxidation was similar to that of HMFCA and FFCA (FIG. 4d). These results showed that HMF could be rapidly converted to the intermediates on the NiCoP surface, suggesting that the oxidation of alcohol and aldehyde groups of HMF on the NiCoP surface could be initiated differently from the other electrodes. In addition, these results also indicated that NiCoP had a large amount of Ni³⁺ on the surface from the beginning at the onset potential compared to the other electrodes (FIG. 10c).

Additionally, the NiCoP electrode showed a different trend in the intermediate formation during FDCA production compared to the other electrodes (FIG. 13). In the NiCoP electrode, FDCA was rapidly generated in proportion to the HMF conversion rate, without retaining DFF and HMFCA (FIG. 13, d). This indicated that HMF oxidation and FDCA generation were performed rapidly on the NiCoP surface. In contrast, significant amounts of DFF and FFCA were retained in the early and late stages of the conversion process in the Ni, NiCo, and NiCoS electrodes (a to c of FIGS. 13). These results were attributed to the low reactivity and intermediate oxidation of HMF.

The enhanced properties of the electrochemical HMF conversion in the NiCoP electrode were analyzed using steady-state LSV (SSLSV) technique.

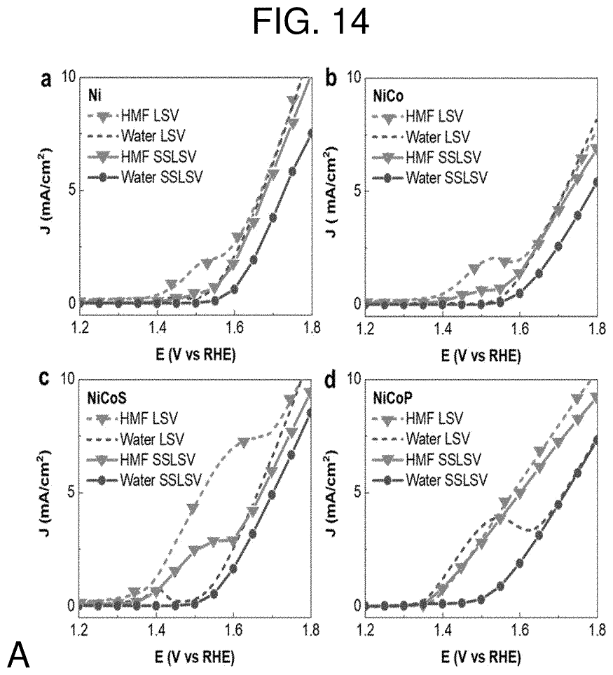

FIG. 14 is a graph showing the LSV and SSLSV analyses results of the catalyst electrodes according to Examples and Comparative Examples of the present invention.

FIG. 14 shows the results of 30 mM HMF (red) and water (black) oxidation using (a) Ni foil, (b) NiCo, (c) NiCoS, and (d) NiCoP foil electrodes in a 0.1 M KOH solution, wherein LSV is indicated by a dotted line, and SSLSV is indicated by a solid line with a symbol. The scan rate of the LSV curve is 10 mV·s⁻¹.

The charge transfer kinetics of the electrode surface was qualitatively evaluated by comparing the LSV curves and the SSLSV curves. The SSLSV curves were plotted by collecting the current densities at the applied potentials when the generated current was stabilized at a constant value. The concentration of dissolved HMF was increased and the HMF solution was stirred to obtain reliable current densities. The SSLSV curves for HMF oxidation using Ni, NiCo, and NiCoS electrodes showed significant differences from the LSV curves (FIGS. 14a to 14c). These results indicated that either the initial rate of surface charge consumption for HMF oxidation reaction was not maintained or the catalytically active Ni ion states for HMF oxidation was not rapidly regenerated on the surface. In contrast, the NiCoP electrode showed almost identical current densities in both the SSLSV and LSV curves for HMF oxidation. The surface leaching charge of the NiCoP electrode can be consumed continuously and rapidly for HMF consumption, intermediate oxidation and/or regeneration of Ni³⁺ state due to the abundantly present Ni³⁺ and easy transition from Ni²⁺ to Ni³⁺.

The large difference between LSV and SSLSV for water oxidation (FIG. 13, d) indicated that a large amount of Ni²⁺ state, which could be immediately converted to Ni³⁺ state, was abundantly present on the NiCoP surface, as compared to the other electrodes (FIGS. 14, a to c). There results show that the sum of Ni²⁺ and Ni³⁺ states of NiCoP was much higher than that of other electrodes, as investigated in the XPS Ni 2p_{3/2} spectrum (FIG. 10, a).

Although the detailed description of the present invention has been described with reference to the preferred embodiments, it will be appreciated by those skilled in the corresponding art or those having ordinary knowledge in the corresponding art that the present invention may be modified and altered in various manners without departing from the spirit and technical scope of the present invention that are set forth in the following claims.

Therefore, the technical scope of the present invention is defined by the appended claims rather than the detailed description.

1. A catalyst compound, which comprises a compound of Chemical Formula 1 below and catalyzes the process of oxidizing 5-hydroxymethylfurfural (HMF) to 2,5-furandicarboxylic acid (FDCA):
NiCoₓP_{y} [Chemical Formula 1]
(wherein x and y are the molar ratio for Ni contained in the catalyst compound, 0<x<1, 0<y<1).

2. The catalyst compound of claim 1, wherein the amount of Ni³⁺ is higher than that of Ni²⁺ in the catalyst compound.

3. A catalyst electrode, which comprises a catalyst compound comprising a compound of Chemical Formula 1 below; and a substrate on which the catalyst compound is provided, and thereby catalyzes the process of oxidizing 5-hydroxymethylfurfural (HMF) to 2,5-furandicarboxylic acid (FDCA):
NiCoₓP_{y} [Chemical Formula 1]
(wherein x and y are the molar ratio for Ni contained in the catalyst compound, 0<x<1, 0<y<1).

4. The catalyst electrode of claim 3, wherein the substrate is at least one selected from the group consisting of a metal foam, a metal foil, carbon paper, and carbon cloth.

5. The catalyst electrode of claim 4, wherein nickel hydroxide is provided on the surface of the substrate.

6. The catalyst electrode of claim 5, wherein the amount of Ni³⁺ is higher than that of Ni²⁺ in the catalyst compound.

7. A method for producing FDCA, comprising: reacting 5-hydroxymethylfurfural (HMF) with a catalyst compound of Chemical Formula 1 below to oxide the 5-hydroxymethylfurfural to 2,5-furandicarboxylic acid (FDCA):
NiCoₓP_{y} [Chemical Formula 1]
(wherein x and y are the molar ratio for Ni contained in the catalyst compound, 0<x<1, 0<y<1).

8. The process of claim 7, wherein the 2,5-furandicarboxylic acid (FDCA) is produced by applying a potential of 1.40 V_{RHE} to 1.60 V_{RHE} to the 5-hydroxymethylfurfural (HMF) and the catalyst compound.

9. The process of claim 8, wherein the oxidation reaction of 5-hydroxymethylfurfural is performed in a basic environment without a base-induced polymerization reaction of the 5-hydroxymethylfurfural.

10. An FDCA production reactor, comprising:
an inlet for introducing 5-hydroxymethylfurfural (HMF);
a catalyst electrode comprising a catalyst compound comprising a compound of Chemical Formula 1 below, and a substrate on which the catalyst compound is provided; and
an outlet for discharging 2,5-furandicarboxylic acid (FDCA) produced after the oxidation reaction of 5-hydroxymethylfurfural (HMF) performed in the catalyst electrode:
NiCoₓP_{y} [Chemical Formula 1]
(wherein x and y are the molar ratio for Ni contained in the catalyst compound, 0<x<1, 0<y<1).

11. The reactor of claim 10, further comprising a power supply for applying a potential to the catalyst electrode.

12. The reactor of claim 10, wherein the substrate is at least one selected from the group consisting of a metal foam, a metal foil, carbon paper, and carbon cloth.

13. The reactor of claim 12, wherein nickel hydroxide is provided on the surface of the substrate.

14. The reactor of claim 13, wherein the amount of Ni³⁺ is higher than that of Ni²⁺ in the catalyst compound.

15. A method for synthesizing a catalyst compound, comprising the steps of:
preparing a NiCo bimetal compound by co-depositing Ni²⁺ and Co²⁺; and
preparing a catalyst compound of Chemical Formula 1 below by reacting the NiCo bimetal compound with a phosphorus compound:
NiCoₓP_{y} [Chemical Formula 1]
(wherein x and y are the molar ratio for Ni contained in the catalyst compound, 0<x<1, 0<y<1).

16. A method for synthesizing a catalyst compound, comprising the steps of:
mixing Ni²⁺, Co²⁺, a phosphorus compound, and a reducing agent; and
preparing a catalyst compound of Chemical Formula 1 below by heat treating the above mixture:
NiCoₓP_{y} [Chemical Formula 1]
(wherein x and y are the molar ratio for Ni contained in the catalyst compound, 0<x<1, 0<y<1).
